(19) United States
(12) Patent Application Publication
(10) Pub. No.: US 2017/0326155 A1
(43) Pub. Date: Nov. 16, 2017

(54) NOVEL PHARMACOLOGICAL THERAPY FOR NEUROMUSCULAR CONTRACTURES

(71) Applicant: Children's Hospital Medical Center, Cincinnati, OH (US)

(72) Inventor: Roger Cornwall, Cincinnati, OH (US)

(21) Appl. No.: 15/590,352

(22) Filed: May 9, 2017

Related U.S. Application Data
(60) Provisional application No. 62/334,691, filed on May 11, 2016.

(51) Int. Cl. A61K 31/565; A61K 47/28

(52) U.S. Cl. CPC A61K 31/565; A61K 47/28; C12Q 2600/106

(57) ABSTRACT

Disclosed are compositions and methods for treatment of an individual having a muscle contracture. The method may include the step of administering nandrolone or a pharmaceutically acceptable salt thereof to the individual in need thereof. The muscle contracture may result from a disorder selected from neonatal brachial plexus injury, cerebral palsy, or a combination thereof. Articles of manufacture containing nandrolone are also disclosed.

### CROSS REFERENCE TO RELATED APPLICATIONS

This application claims priority to and the benefit of U.S. Provisional Patent Application Ser. No. 62/334,691, filed May 11, 2016, to Cornwall, entitled “Novel Pharmacological Therapy for Neuromuscular Contractures,” the contents of which are incorporated by reference in its entirety.

### BACKGROUND

Neonatal brachial plexus injury (NBPI) and cerebral palsy (CP) are the two most common causes of paralysis in children. As more and younger pre-term infants survive, the rate of CP is increasing. However, it is not muscle weakness that causes the greatest physical impairment in these children, but rather secondary, progressive muscle contractures that severely limit joint flexibility. These contractures in turn lead to progressive skeletal deformities that further limit function. These contractures and deformities cannot be cured with any existing therapies, and they place an enormous burden on the patients, caregivers, and healthcare system. For over 100 years, clinicians have treated the secondary contractures from NBPI and CP as mechanical problems, but without success.

### BRIEF SUMMARY

Disclosed are compositions and methods for treatment of an individual having a muscle contracture. The method may include the step of administering nandrolone or a pharmaceutically acceptable salt thereof to the individual in need thereof. The muscle contracture may result from a disorder selected from neonatal brachial plexus injury, cerebral palsy, or a combination thereof. Articles of manufacture containing nandrolone are also disclosed.

### BRIEF DESCRIPTION OF THE DRAWING

FIG. 1. Nandrolone (single systemic dose) given following NBPI lessened the severity of shoulder contractures at 4 weeks post NBPI.

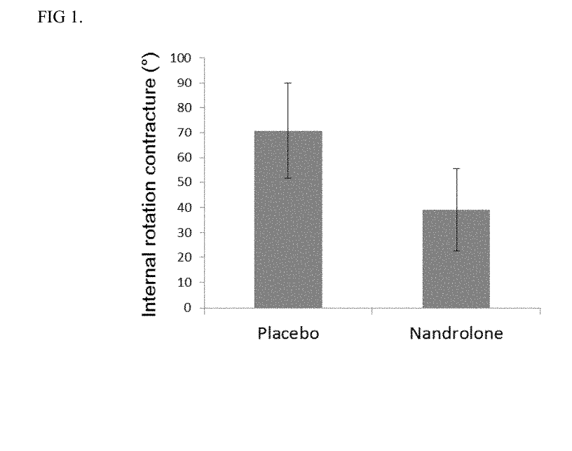

### DETAILED DESCRIPTION OF THE INVENTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms and expressions used herein have the ordinary meaning as is accorded to such terms and expressions with respect to their corresponding respective areas of inquiry and study except where specific meanings have otherwise been set forth herein.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, for example, within 5-fold, and or for example, within 2-fold, of a value. Where particular values are described in the application and claims, unless otherwise stated the term “about” meaning within an acceptable error range for the particular value should be assumed.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The term “therapeutically effective amount,” as used herein, refers to any amount of a compound which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

The terms “treat,” “treating” or “treatment,” as used herein, refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

The term “pharmaceutically acceptable,” as used herein, refers a material, such as a carrier or diluent, which does not abrogate the biological activity or properties of the compounds described herein. Such materials are administered to an individual without causing undesirable biological effects or interacting in a deleterious manner with any of the components of the composition in which it is contained.

The term “pharmaceutically acceptable salt,” as used herein, refers to a formulation of a compound that does not cause significant irritation to an organism to which it is administered and does not abrogate the biological activity and properties of the compounds described herein.

The terms “composition” or “pharmaceutical composition,” as used herein, refers to a mixture of at least one compound, such as the compounds of Formula (I) provided herein, with at least one and optionally more than one other pharmaceutically acceptable chemical components, such as carriers, stabilizers, diluents, dispersing agents, suspending agents, thickening agents, and/or excipients.

The term “carrier” applied to pharmaceutical compositions of the disclosure refers to a diluent, excipient, or vehicle with which an active compound (e.g., dextromethorphan) is administered. Such pharmaceutical carriers can be sterile liquids, such as water, saline solutions, aqueous dextrose solutions, aqueous glycerol solutions, and oils, including those of petroleum, animal, vegetable, or synthetic origin, such as peanut oil, soybean oil, mineral oil, sesame oil and the like. Suitable pharmaceutical carriers are described in “Remington's Pharmaceutical Sciences” by E. W. Martin, 18th Edition.

Applicant has found a novel method by which an individual in need thereof can be treated pharmacologically to prevent contracture formation by correcting the underlying muscular pathophysiology caused by the neonatal neurologic problem. Neonatal brachial plexus injury (NBPI) and cerebral palsy (CP) are the two most common causes of neuromuscular dysfunction in childhood, occurring in a combined 5 per 1,000 live births. Both conditions lead to permanent and progressive muscle contractures that limit joint range of motion. These contractures are a major source of disability and are the most common indication for rehabilitative and surgical intervention. However, existing contracture treatments used in either condition cannot restore normal joint motion, and can cause further loss of function by weakening the muscles responsible for the contractures. Furthermore, the muscular contractures alter the physical forces on the developing skeleton, leading to dysplasia and dislocation of joints such as the shoulder in NBPI and the hip in CP. Therefore, once contractures have developed, a progressive and currently incurable cascade of musculoskeletal dysfunction begins. We are thus in critical need of an effective method to prevent contracture formation in neonatal-onset neuromuscular disorders by understanding and correcting the underlying muscular pathophysiology. In the current proposal, we seek to meet that need by translating our research regarding contracture pathophysiology into a novel pharmacological contracture prevention strategy through the repurposing of existing medications.

By developing a mouse model of NBPI, we have demonstrated that contractures result from impaired longitudinal growth of muscles denervated during a critical window of neonatal muscle development. We thus currently hold intellectual property regarding muscle growth modulation as a novel strategy to prevent and treat neonatal neuromuscular contractures. Even though the precise molecular mechanisms modulating neonatal muscle growth and how they are disrupted by NBPI are not yet elucidated, we have an opportunity now with our clinically relevant NBPI model to test the repurposing of existing medications known to promote muscle growth as a novel contracture prevention strategy. Neonatal muscle growth involves both addition of myonuclei to the growing myofiber (myonuclear accretion) and protein synthesis. We have demonstrated that NBPI disrupts satellite cell (muscle stem cell) behavior, although myonuclear accretion is not wholly prevented following NBPI. We have also demonstrated that NBPI reduces muscle length, mass and volume through inadequate generation of muscle proteins (sarcomerogenesis), although with normal transcription of sarcomeric protein genes, suggesting reduced protein translation. We have preliminarily demonstrated lessened contracture severity following NBPI with neonatal treatment with nandrolone, an anabolic steroid that promotes satellite cell differentiation as well as protein translation. However, the dosage and safety profile of nandrolone in the neonatal period has not been investigated. Moreover, impaired muscle growth is not the only muscular abnormality in neonatal-onset neuromuscular contractures, where muscle fibrosis is also a prominent finding, both in NBPI and CP. The angiotensin II receptor antagonist, losartan, has been shown to improve adult skeletal muscle regeneration, presumably by inhibiting TGFr3 signaling-induced fibrosis and by driving satellite cell differentiation. Losartan also increases Akt/mTOR signaling, the master driver of muscle protein translation, but it has not been tested for this use in pediatric neuromuscular conditions.

In one aspect, a method of treating an individual having a muscle contracture is disclosed. The method may comprise the step of administering nandrolone or a pharmaceutically acceptable salt thereof to the individual in need thereof. Nandrolone, also known as 19-nortestosterone or 19-norandrostenolone, is an anabolic steroid available from, for example, Sigma Aldrich, having the following structure:

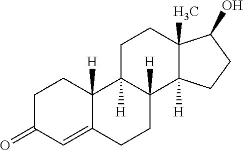

In one aspect, the muscle contracture may result from a disorder selected from neonatal brachial plexus injury, cerebral palsy, or a combination thereof.

In one aspect, the administration step may occur during a period of from birth to about six months of age, or from about one month of age to about three months of age, or from about one month of age to about six months of age.

In one aspect, the administration step occurs during a period of from within about one month of birth to about three months of age.

In one aspect, the administration step may be carried out until recovery of neurological function, wherein the recovery of neurological function is determined by spontaneous nerve healing or nerve recovery, in the absence of muscle contractures.

In one aspect, the muscle contracture may be a shoulder muscle contracture. The administration may be carried out until the shoulder muscle contracture is improved as measured by internal rotation contracture (degrees) as compared to pre-treatment muscle contracture.

In one aspect, the muscle contracture is elbow muscle contracture. In this aspect, the administration step may be carried out until the elbow muscle contracture is improved as measured by internal rotation contracture (degrees) as compared to pre-treatment muscle contracture.

In one aspect, the administration step may be carried out for a period of time sufficient to reduce the severity of, reduce the progression of, reduce the onset of, or prevent muscle contractures in the first year of life of said individual.

In one aspect, the administration step may be carried out for a period of time sufficient to preserve longitudinal muscle growth during a neonatal period, wherein the neonatal period is approximately the first three to six months of life.

In one aspect, the administration step may be carried out for a period of time sufficient to reverse a decreased range of motion resulting from neonatal brachial plexus injury and/or cerebral palsy.

In one aspect, a method of improving impaired joint flexibility in an individual having neonatal brachial plexus injury and/or cerebral palsy, wherein said impaired joint flexibility results from secondary muscle contractures, is disclosed. In this aspect, the method may comprise the step of administering nandrolone or a pharmacologically acceptable salt thereof to the individual.

In one aspect, a method of preserving longitudinal muscle growth during neonatal muscle development in an individual having neonatal brachial plexus injury and/or cerebral palsy is disclosed. In this aspect, the method may comprise the step of administering nandrolone or a pharmacologically acceptable salt thereof.

In one aspect, the muscle growth and contracture prevention may be achieved via preservation of afferent and/or sympathetic innervation of the muscle.

In one aspect, an article of manufacture is disclosed. The article of manufacture may comprise a container comprising a label and a composition comprising nandrolone or a pharmaceutically acceptable salt thereof and a pharmaceutically acceptable carrier, wherein the label indicates that the composition is to be administered to an individual having or at risk of developing muscle contractures. The individual may be diagnosed with neonatal brachial plexus injury and/or cerebral palsy. The article of manufacture may further comprise a means for delivery of said composition to an individual.

Dosage

In one aspect, an agent disclosed herein may be present in an amount of from about 0.5% to about 95%, or from about 1% to about 90%, or from about 2% to about 85%, or from about 3% to about 80%, or from about 4%, about 75%, or from about 5% to about 70%, or from about 6%, about 65%, or from about 7% to about 60%, or from about 8% to about 55%, or from about 9% to about 50%, or from about 10% to about 40%, by weight of the composition.

The compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms all utilizing dosage forms well known to those of ordinary skill in the pharmaceutical arts. The compositions may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen.

A dosage regimen will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect. The effective amount of a drug required to prevent, counter, or arrest progression of a symptom or effect of a muscle contracture can be readily determined by an ordinarily skilled physician

The pharmaceutical compositions may include suitable dosage forms for oral, parenteral (including subcutaneous, intramuscular, intradermal and intravenous), transdermal, sublingual, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. Oral preparations include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. For topical or nasal administration, penetrants or permeation agents that are appropriate to the particular barrier to be permeated are used in the formulation. Such penetrants are generally known in the art. The pharmaceutical compositions are prepared by conventional techniques appropriate to the desired preparation containing appropriate amounts of the active ingredient, that is, one or more of the disclosed active agents or a pharmaceutically acceptable salt thereof according to the invention.

The dosage of an agent disclosed herein used to achieve a therapeutic effect will depend not only on such factors as the age, weight and sex of the patient and mode of administration, but also on the degree of inhibition desired and the potency of an agent disclosed herein for the particular disorder or disease concerned. It is also contemplated that the treatment and dosage of an agent disclosed herein may be administered in unit dosage form and that the unit dosage form would be adjusted accordingly by one skilled in the art to reflect the relative level of activity. The decision as to the particular dosage to be employed (and the number of times to be administered per day) is within the discretion of the physician, and may be varied by titration of the dosage to the particular circumstances of this invention to produce the desired therapeutic effect.

Kits

Kits are also provided. In one aspect, a kit may comprise or consist essentially of agents or compositions described herein. The kit may be a package that houses a container which may contain a composition comprising an oxime or pharmaceutically acceptable salt thereof as disclosed herein, and also houses instructions for administering the agent or composition to a subject. In one aspect, a pharmaceutical pack or kit is provided comprising one or more containers filled with one or more composition as disclosed herein. Associated with such container(s) can be various written materials such as instructions for use, or a notice in the form prescribed by a governmental agency regulating the manufacture, use or sale of pharmaceuticals or biological products, which notice reflects approval by the agency of manufacture, use, or sale for human administration.

As there may be advantages to mixing a component of a composition described herein and a pharmaceutically acceptable carrier, excipient or vehicle near the time of use, kits in which components of the compositions are packaged separately are disclosed. For example, the kit can contain an active ingredient in a powdered or other dry form in, for example, a sterile vial or ampule and, in a separate container within the kit, a carrier, excipient, or vehicle, or a component of a carrier, excipient, or vehicle (in liquid or dry form). In one aspect, the kit can contain a component in a dry form, typically as a powder, often in a lyophilized form in, for example, a sterile vial or ampule and, in a separate container within the kit, a carrier, excipient, or vehicle, or a component of a carrier, excipient, or vehicle. Alternatively, the kit may contain a component in the form of a concentrated solution that is diluted prior to administration. Any of the components described herein, any of the carriers, excipients or vehicles described herein, and any combination of components and carriers, excipients or vehicles can be included in a kit.

Optionally, a kit may also contain instructions for preparation or use (e.g., written instructions printed on the outer container or on a leaflet placed therein) and one or more devices to aid the preparation of the solution and/or its administration to a patient (e.g., one or a plurality of syringes, needles, filters, tape, tubing (e.g., tubing to facilitate intravenous administration) alcohol swabs and/or the Band-Aid® applicator). Compositions which are more concentrated than those administered to a subject can be prepared. Accordingly, such compositions can be included in the kits with, optionally, suitable materials (e.g., water, saline, or other physiologically acceptable solutions) for dilution. Instructions included with the kit can include, where appropriate, instructions for dilution.

In other embodiments, the kits can include pre-mixed compositions and instructions for solubilizing any precipitate that may have formed during shipping or storage. Kits containing solutions of Compound I, or a pharmaceutically acceptable salt thereof, and one or more carriers, excipients or vehicles may also contain any of the materials mentioned above (e.g., any device to aid in preparing the composition for administration or in the administration per se). The instructions in these kits may describe suitable indications (e.g., a description of patients amenable to treatment) and instructions for administering the solution to a patient.

### Examples

Experimental Model: Unilateral brachial plexus injuries are surgically created at 5 days of age in mice. The immediate paralysis mimics that of the human condition, and can be modulated by different surgical techniques of nerve injury. Within 4 weeks after NBPI, muscle contractures form at the shoulder and elbow, replicating the human contractures. (Not shown.) Muscles can be harvested for analysis of growth and cellular behavior. The use of unilateral injuries preserves the contralateral limb as a control, minimizing the number of mice required.

Using our mouse model of NBPI, we have demonstrated that neonatal denervation leads to impaired longitudinal muscle growth, a process that requires myonuclear accretion from muscle stem cells (satellite cells) and synthesis of proteins required for sarcomerogenesis. The relative importance of these two processes and the molecular mechanisms that control them are not well understood in normal muscle growth. Nonetheless, we have demonstrated abnormal behavior of satellite cells following denervation, with a relative deficit of differentiated satellite cells, although myonuclear accretion is not wholly prevented. Furthermore, we have demonstrated substantially reduced sarcomerogenesis, as evidenced by overstretched sarcomeres, indicating fewer than normal sarcomeres in series, as well as reduced muscle protein volume and mass. Importantly, this sarcomere overstretch is also a hallmark of muscle contractures in CP. The reduction in muscle protein is not accompanied by a reduction in transcription of sarcomeric protein genes, suggesting reduced protein translation.

Applicant has found that nandrolone, decreased the severity of the shoulder contracture compared to controls in a preliminary experiment.

### REFERENCES

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of treating an individual having a muscle contracture, comprising the step of administering nandrolone or a pharmaceutically acceptable salt thereof to an individual in need thereof.

2. The method of claim 1, wherein said muscle contracture results from a disorder selected from neonatal brachial plexus injury, cerebral palsy, or a combination thereof.

3. The method of claim 1, wherein said administration step occurs during a period of from birth to about six months of age.

4. The method of claim 1, wherein said administration step occurs during a period of from within about one month of birth to about three months of age.

5. The method of claim 1, wherein said administration step is carried out until recovery of neurological function, wherein said recovery of neurological function is determined by spontaneous nerve healing or nerve recovery, in the absence of muscle contractures.

6. The method of claim 1, wherein said muscle contracture is a shoulder muscle contracture, and wherein said administration is carried out until said shoulder muscle contracture is improved as measured by internal rotation contracture (degrees).

7. The method of claim 1, wherein said muscle contracture is elbow muscle contracture, and wherein said administration is carried out until said elbow muscle contracture is improved as measured by internal rotation contracture (degrees).

8. The method of claim 1, wherein said administration step is carried out for a period of time sufficient to reduce the severity of, reduce the progression of, reduce the onset of, or prevent muscle contractures in the first year of life of said individual.

9. The method of claim 1, wherein said administration step is carried out for a period of time sufficient to preserve longitudinal muscle growth during a neonatal period, wherein said neonatal period is the first three to six months of life.

10. The method of claim 1, wherein said administration step is carried out for a period of time sufficient to reverse a decreased range of motion resulting from neonatal brachial plexus injury and/or cerebral palsy.

11. A method of improving impaired joint flexibility in an individual having neonatal brachial plexus injury and/or cerebral palsy, wherein said impaired joint flexibility results from secondary muscle contractures, comprising the step of administering nandrolone or a pharmacologically acceptable salt thereof to said individual.

12. The method of claim 11, wherein said improvement results from preserving longitudinal muscle growth during neonatal muscle development.

13. The method of claim 12, wherein said muscle growth and contracture prevention is achieved via preservation of afferent and/or sympathetic innervation of the muscle.

14. An article of manufacture comprising:
a. a container comprising a label; and
b. a composition comprising nandrolone or a pharmaceutically acceptable salt thereof and a pharmaceutically acceptable carrier;
wherein the label indicates that the composition is to be administered to an individual having or at risk of developing muscle contractures.

15. The article of manufacture of claim 14, wherein said individual is diagnosed with neonatal brachial plexus injury and/or cerebral palsy.

16. The article of manufacture of claim 14, further comprising a means for delivery of said composition to an individual.
